(19) United States
(12) Patent Application Publication
(10) Pub. No.: US 2026/0207176 A1
(43) Pub. Date: Jul. 23, 2026

(54) System and Method for Analysis of Endoscopic Imaging

(71) Applicant: Vanquish Bio, Inc., Longwood, FL (US)

(72) Inventor: Bryan ALLINSON, Longwood, FL (US)

(21) Appl. No.: 19/453,495

(22) Filed: Jan. 20, 2026

Related U.S. Application Data
(60) Provisional application No. 63/747,060, filed on Jan. 19, 2025.

(51) Int. Cl. A61B 8/00; A61B 1/00; A61B 8/08; A61B 8/12

(52) U.S. Cl. CPC A61B 8/5223; A61B 1/000096; A61B 8/085; A61B 8/12; A61B 8/463; A61B 8/481; A61B 8/485; A61B 8/488

(57) ABSTRACT

A system and method for artificial intelligence (AI)-assisted medical diagnostics. A real-time endoscopic ultrasound (EUS) video stream of a patient's organ, such as the pancreas, is received at a computing device. The EUS video stream comprises a plurality of imaging modalities, including at least grey-scale data and elastography data representing tissue stiffness. One or more processors execute an AI model, which processes the video stream to identify, segment, and characterize a region of interest. A visual output is generated for display on a user interface in real-time, the output comprising the EUS video stream with a graphical overlay that includes a visual indicator delineating the segmented region and a classification label corresponding to the characterization, thereby providing real-time clinical decision support.

### TECHNICAL FIELD

The present invention relates generally to the field of medical diagnostic systems, and more specifically, to computer-implemented systems and methods for analyzing medical imaging data using artificial intelligence to detect and characterize tissue abnormalities.

### BACKGROUND

Pancreatic cancer is one of the most lethal forms of cancer, primarily due to its late stage of diagnosis. Symptoms often do not appear until the disease is advanced, and the pancreas's location deep within the body makes it difficult to detect tumors during routine physical examinations.

Endoscopic ultrasound (EUS) is a specialized medical procedure used for obtaining detailed images of the digestive tract and nearby organs, including the pancreas. In an EUS procedure, a thin, flexible tube (an endoscope) with a small ultrasound probe at its tip is guided through a patient's digestive tract. This allows for high-resolution imaging from inside the body, providing a closer view than traditional external ultrasound. EUS can be effective for the early detection of pancreatic cancer.

However, the effectiveness of EUS is highly dependent on the skill and experience of the clinician performing the procedure. The interpretation of EUS images is a complex task, and even a trained clinician may miss subtle features indicative of malignancy. Furthermore, a definitive diagnosis often requires a fine-needle aspiration or biopsy (FNA/FNB) to obtain a tissue sample. This confirmatory procedure carries inherent risks, such as bleeding, and clinicians may be hesitant to perform a biopsy unless they are highly confident that a detected lesion is suspicious. This creates a high threshold for diagnosis and can lead to missed opportunities for early intervention.

### SUMMARY

The disclosed implementations provide systems and methods for real-time, AI-assisted analysis of medical imaging to overcome the limitations of conventional diagnostic procedures. The disclosed implementations provide a clinical decision support tool for use during endoscopic ultrasound (EUS) procedures.

In one aspect, a computer-implemented method for real-time analysis of pancreatic tissue is provided. The method comprises receiving a real-time EUS video stream of a patient's pancreas. This video stream is multi-modal, including at least grey-scale data and elastography data, which provides information about tissue stiffness. An artificial intelligence (AI) model processes this video stream to automatically identify a region of interest, segment its boundaries from surrounding tissue, and generate a characterization of the tissue. A visual output, including the live video stream with a graphical overlay showing the segmented region and a classification label, is then generated for display on a user interface. This provides the clinician with immediate, actionable feedback.

In another aspect, a system for performing this method is provide. The system comprises one or more processors and a memory storing instructions that, when executed, cause the system to perform the steps of receiving the multi-modal EUS video stream, processing it with an AI model to identify, segment, and characterize pancreatic tissue, and generating the real-time visual output with graphical overlays.

The AI model can be deployed in a cloud-computing environment, allowing the system to be accessed via a browser on a client device in the procedure room. This enables widespread, standardized use across diverse clinical settings, including rural and underserved areas.

The system can integrate additional data sources to refine its analysis. This can include other EUS imaging modalities like color doppler or contrast-enhanced imaging. The system may also access non-imaging patient data from an electronic medical record (EMR), such as laboratory results, pathology reports, genomic data (e.g., KRAS, BRCA1/2 mutations), and patient history to provide a more holistic analysis.

Based on its characterization of tissue, the system can also generate a recommendation for a subsequent clinical action, such as performing a fine-needle biopsy on a suspicious lesion, thereby guiding the clinician's next steps. The system is also designed to be a learning system, capable of receiving feedback from clinicians and being re-trained with new data to continuously improve its diagnostic accuracy.

### BRIEF DESCRIPTION OF THE DRAWINGS

The accompanying drawings, which are incorporated in and constitute a part of this specification, illustrate embodiments of the invention and, together with the description, serve to explain the principles of the invention.

FIG. 1 is a schematic block diagram illustrating an exemplary system architecture for an AI-assisted medical diagnostic system, in accordance with disclosed implementations.

FIG. 2 is a flowchart illustrating the steps of a computer-implemented method for real-time analysis of endoscopic imaging,, in accordance with disclosed implementations.

FIG. 3 illustrates a graphical user interface displaying the visual output of the system, in accordance with disclosed implementations.

FIG. 4 is a block diagram illustrating the integration of multi-modal data sources for analysis by the AI engine, in accordance with one embodiment of the invention.

### DETAILED DESCRIPTION

There is a significant need for a system that can assist clinicians in interpreting EUS images, standardize the diagnostic process, enhance detection sensitivity, and provide objective data to support the critical decision of whether to proceed with a biopsy. An improved system would augment the clinician's expertise, reduce diagnostic uncertainty, and ultimately improve patient outcomes through earlier and more accurate detection of pancreatic diseases.

The following description refers to the accompanying drawings in which the same numbers in different drawings represent the same or similar elements unless otherwise represented. The implementations set forth in the following description are merely examples of systems and methods consistent with aspects related to the invention as recited in the appended claims.

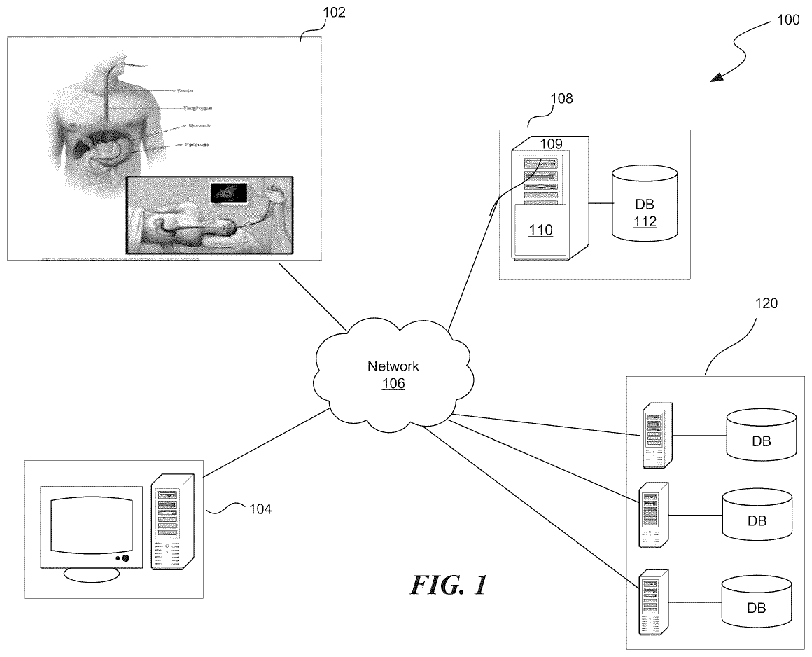

FIG. 1 is a schematic block diagram illustrating an exemplary system architecture 100 of disclosed implementation. The system 100 includes an Endoscopic Ultrasound system (EUS) 102, a client computing device 104, a network 106, and a remote cloud platform 108.

The EUS system 102 can be a conventional medical apparatus used by a clinician to perform an EUS procedure. It typically includes an endoscope with an ultrasound probe at its distal end, a processor, and a display. The EUS system 102 generates a real-time video stream of a patient's internal organs, such as the pancreas. In the disclosed implementations, the EUS system 102 is capable of generating multi-modal image data, which can include standard B-mode (grey-scale) ultrasound data as well as elastography data. Elastography is a mode that measures and depicts tissue stiffness, which provides a mechanism for differentiating hard, inelastic tumor tissue from soft, elastic normal pancreatic tissue. The EUS system 102 may also generate other modalities like color doppler or contrast-enhanced ultrasound data.

The client computing device 104 can be a local device, such as a laptop computer, a desktop computer, or a tablet, located in or near the procedure room. The client device 104 is operably connected to the EUS system 102 to receive the real-time EUS video stream. The client device 104 includes a display, a processor, and a network interface. Its primary functions are to transmit the video stream to the cloud platform 108 and to display the processed results received back from the platform for the clinician to view. The system can be in communication with various external systems 120 (such as EMR systems).

The network 106 represents a data communication network, such as the internet or a local area network (LAN), which facilitates communication between the client device 104 and the cloud platform 108. The cloud platform 108 comprises one or more remote servers, hosts an AI model 110 and is connected to a database 112. The AI model 110 comprises one or more machine learning models, such as a convolutional neural network (CNN), a recurrent neural network (RNN), or a hybrid model, that has been trained to analyze EUS image data. The AI model 110 is configured to perform the tasks of object detection, segmentation, and classification on the received video stream (as described in greater detail below). The database 112 can store one or more trained AI model(s) and hyperparameters thereof, as well as a vast, de-identified dataset used for training, validation, and retraining (also described in more detail below). This dataset can include annotated EUS images and additional confirmatory ground-truth data, such as pathology reports, cytology results, and clinical follow-up information.

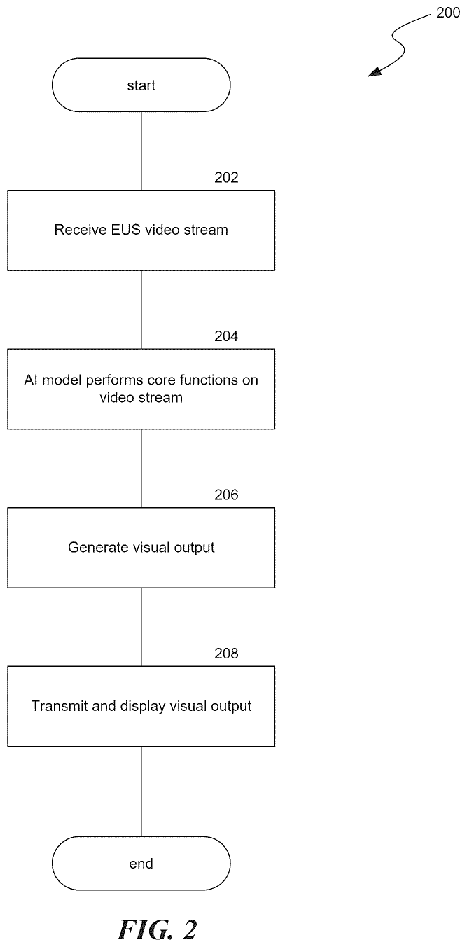

FIG. 2 is a flowchart illustrating the steps of a method 200 for real-time analysis of EUS imaging. At step 202, the system receives the real-time, multi-modal EUS video stream. As described above, this stream includes at least grey-scale and elastography data.

At step 204, the video stream is processed by the AI model 110. In the cloud-based implementation, this involves the client device 104 transmitting the data stream over the network 106 to the AI model 110 on the cloud platform 108. The AI model 110 performs its core analytical functions on the video frames. This includes:
Identification: The AI model analyzes the image features to detect the presence and location of a potential region of interest (ROI), such as a solid mass or a cystic lesion, within the pancreas.
Segmentation: Once an ROI is identified, the model delineates its precise boundaries, creating a segmentation mask that separates the lesion from surrounding healthy tissue.
Characterization: The model then analyzes the features within the segmented ROI across the multiple imaging modalities. For example, it analyzes texture and shape from the grey-scale data and, critically, analyzes stiffness values from the elastography data. Based on this multi-modal analysis, it generates a characterization of the tissue, classifying it as, for example, a “solid tumor,” “cyst,” or “normal.”

At step 206, a visual output is generated. This output consists of the original EUS video frame combined with a graphical overlay. The overlay contains a visual indicator corresponding to the segmentation (e.g., a colored bounding box or a highlighted mask) and a text-based classification label.

At step 208, the visual output is transmitted back to the client device 104 and displayed on its user interface in real-time or near-real-time. This provides the clinician performing the EUS procedure with immediate, intuitive, and actionable feedback to guide their diagnostic assessment during and after the EUS procedure.

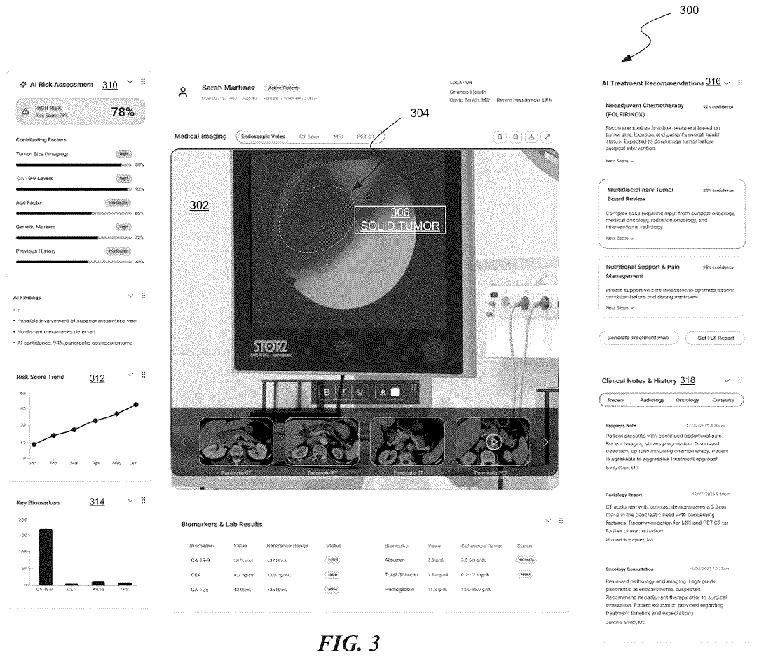

FIG. 3 shows an exemplary graphical user interface (GUI) 300 as it would be displayed on the client device 104. The GUI 300 includes a display area 302 that presents the live EUS video feed. Within the video feed, a region of interest, such as a potential tumor in the pancreas, is visible. Superimposed on the video feed is a graphical overlay generated by the system. This overlay includes a visual indicator 304, such as a colored segmentation mask or bounding box, that precisely delineates the boundaries of the region of interest 306 as determined by the AI model. The overlay can also includes a classification label 306, such as “SOLID TUMOR,” “CYSTIC LESION,” or “NORMAL,” which corresponds to the characterization generated by the AI model. This integrated display allows a clinician to see both the raw image data and the evaluation by the AI model simultaneously, enhancing the clinician's ability to make an informed clinical judgment.

GUI 300 can also include risk assessment field 310, risk score trend field 312, biomarkers field 314, treatment recommendations field 316 and clinical notes field 318. Risk assessment field 310 presents quantitative or qualitative information regarding the likelihood of disease presence, progression, or adverse outcomes for the patient. The data may be derived from the AI analysis of imaging, clinical history, and other integrated sources, and could include probability scores, risk categories (e.g., “low,” “moderate,” “high”), or relevant explanatory factors (such as lesion size, patient age, and comorbidities). This helps clinicians quickly understand how concerning a detected lesion or abnormality might be. Risk trend field 312 displays how the patient's risk score has changed over time, often visualized as a graph or timeline. By tracking these trends, such as increases or decreases in risk following interventions or changes in clinical status, clinicians can monitor disease progression, response to treatment, and the impact of lifestyle or medical changes. Biomarkers field 314 displays measurable indicators (biomarkers) of biological processes, disease states, or responses to therapy. in the context of EUS imaging and AI analysis. For example, this field might display relevant molecular, genetic, or protein markers (such as CA 19-9 for pancreatic cancer, BRCA mutations, or elastography-derived stiffness measurements). Such data can help refine diagnosis, assess prognosis, and guide personalized treatment strategies.

Treatment Recommendations field 316 generates actionable recommendations for next steps based on the characterization determined by the AI model 110 and displayed in label 306. These may include suggestions like “Recommend EUS-guided fine-needle biopsy,” “Schedule follow-up imaging in 3 months,” or “Refer to oncology specialist.” Such guidance moves the system from merely providing diagnostic support to actively informing clinical decision-making, helping streamline care pathways and improve patient outcomes. Clinical history field 318 displays documentation of observations, interpretations, and plans by the clinician. Notes may include contextual information about the case, rationale for decisions, or commentary on the AI-generated findings and recommendations. Integrating clinical notes within the GUI ensures that the full narrative of the patient's care is captured alongside structured data, supporting communication, compliance, and continuity of care.

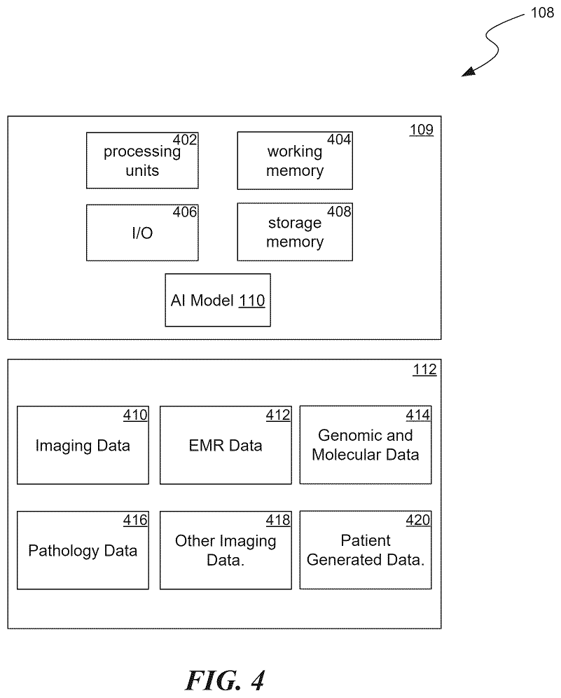

FIG. 4 is a block diagram illustrating the, multi-modal data integration capabilities of the system illustrated in FIG. 1. Server 109 includes processing units 402, working memory 404, I/O interface 406 and storage memory 408. As noted above, AI model 110 executes on server 109 and is architected to receive and process data from a wide variety of sources to generate its output. These sources, treated as independent variables, include:
Real-Time Imaging Data stored in image data module 410: The primary EUS video stream, including grey-scale, elastography, and other modes as previously described.
EMR Data stored in EMR data module 412: Data extracted from a patient's Electronic Medical Record, which can be both structured (e.g., demographics, billing codes, lab values) and unstructured (e.g., text from physician notes, radiology reports).
Genomic & Molecular Data stored in genomic and molecular data module 414: Information such as germline genetic testing results (e.g., BRCA1/2 mutations), somatic tumor profiling from NGS panels, and liquid biopsy data (e.g., circulating tumor DNA (ctDNA)).
Pathology Data stored in pathology data module 416: Digital images from histology or cytology slides, and text-based pathology reports.
Other Imaging Data stored in other imaging data module 412: Static or video data from other imaging modalities like CT, MRI, and PET scans.
Patient-Generated Data stored in patient generated data module 414: Data from wearable devices (e.g., continuous glucose monitors, activity trackers) and lifestyle/behavioral data (e.g., smoking status, diet).

Other non-imaging patient data can encompass a variety of information types that can be used for comprehensive diagnostic assessment and clinical decision-making. The key categories of non-imaging data include:
Laboratory Results: These are quantitative and qualitative measurements obtained from blood, urine, or other bodily fluids. Common laboratory tests relevant to pancreatic disease include liver function tests (ALT, AST, bilirubin), pancreatic enzymes (amylase, lipase), glucose levels, tumor markers (such as CA 19-9), and complete blood counts. These results can indicate organ dysfunction, inflammation, or the presence of malignancy.
Pathology Reports: These documents summarize the findings from tissue samples obtained via biopsy or surgical procedures. Pathology reports detail the microscopic characteristics of cells and tissues, such as the presence of cancerous cells, tumor grade, and margins. They are central to confirming diagnoses and guiding treatment plans.
Genomic Data: Genomic information refers to the results of genetic testing for mutations commonly associated with pancreatic diseases, such as KRAS and BRCA1/2 genes. These mutations can influence disease risk, prognosis, and the selection of targeted therapies. Genomic data may be obtained from specialized laboratories following blood draws or tissue collection.
Patient History: This encompasses the patient's medical, surgical, family, and social history. Relevant details include previous diagnoses, treatments, risk factors (such as smoking or alcohol use), family history of cancer, and symptoms reported over time. Patient history is typically gathered through clinician interviews, questionnaires, and review of prior medical records.

Most non-imaging data can be accessed through the hospital or clinic's EMR platform. EMRs aggregate laboratory results, pathology reports, genetic test results, and documented patient histories in a digital format. Authorized clinicians can retrieve this information using patient identifiers, ensuring secure and efficient access. Laboratory results and genomic data are usually obtained by ordering tests through the EMR or directly from the laboratory. Specimens are collected (blood, tissue, urine), processed, and results are uploaded to the EMR or provided as digital or printed reports. Pathology reports are generated after tissue samples are analyzed by pathologists. Once finalized, these reports are entered into the EMR and become accessible to treating clinicians. Patient history can be gathered during clinical encounters, where clinicians document relevant information directly into the EMR. Additional details may be collected via standardized questionnaires or patient-provided health portals. By integrating these diverse non-imaging data sources, the system can provide a more holistic and precise analysis, leveraging both objective test results and contextual patient information to support diagnosis and clinical decision-making.

The AI model 110 processes this integrated dataset to produce its output, which can be a real-time characterization, a risk score, and/or a treatment recommendation. The system also includes a feedback and retraining loop to allow for clinician feedback on the output of AI model 110, as well as the ingestion of new ground-truth data (e.g., a definitive pathology result after a biopsy), to be fed back into the system. This new data can be used to periodically retrain and update the AI model 110, allowing the system to continuously learn and improve its performance over time. Further, the system may use the dataset to generate new patient documents (by applying a Large Language Model (LLM) or the like), such as a cancer survivorship report, which can be stored in the patient's EMR for longitudinal tracking and compliance with accreditation standards.

The disclosed implementations can include a distributed system architecture designed for high performance, security, and scalability. As noted above in the discussion of FIG. 1, the components of the system include the client computing device 104, and the cloud platform 108. More detailed examples of technical implementations of these components are provided below.

The client computing device 104 can be the primary point of interaction for the clinician within the procedural environment and can serv as the bridge between the EUS system 102 and the cloud platform 108. The client computing device 104 can receive the raw, multi-modal video stream from the Endoscopic Ultrasound (EUS) system, transmit the video stream securely to the Cloud Platform for real-time analysis, receive the processed results (e.g., graphical overlays and classifications) and render the combined visual output—the original video feed plus the AI-generated overlays—on a graphical user interface (GUI) for the clinician.

The client computing device 104 can be implemented on a range of hardware, including a dedicated medical-grade workstation, a high-performance laptop computer, or a tablet device. For example, the device can include a multi-core CPU (e.g., Intel Core i7/i9 or AMD Ryzen 7/9) and 16 GB RAM to handle high-resolution video streams without latency. A high-speed network interface card (e.g., Gigabit Ethernet or Wi-Fi 6/6E) can be provided in the device for low-latency transmission of the video stream and a high-resolution monitor can be included for rendering medical images.

The user interface can be developed as a web application running in a modern browser (e.g., Chrome, Edge) or as a native desktop application (e.g., using Electron or Qt). A web-based implementation can leverage modern frontend frameworks (e.g., React, Vue.js, or Angular) for building a responsive and modular GUI. A component-based architecture allows for flexibility and easy updates. The client computing device can interface with the EUS system's video output, typically via HDMI, SDI, or a proprietary vendor protocol. A video capture card may be used to digitize the signal. For real-time video streaming, a low-latency protocol like WebSockets or a custom UDP-based protocol can be used preferred. For less latency-sensitive commands or metadata, HTTPS-based RESTful APIs or gRPC can be used. The processed overlay data can be received from the cloud via WebSockets or a dedicated channel to ensure synchronization with the live video feed.

The raw video stream can be packetized. Each packet could be a JSON object containing a base64-encoded image frame and associated metadata. An example data structure is set forth below:
′′′json
{
“timestamp”: “2024-10-27t10:30:01.123z”,
“frame_id”: 12345,
“modality”: “elastography”,
“frame_data”: “iVBORw0KGgoAAAANSUhEUg...”
}
′′′

Data received from the cloud can also be a JSON object defining the overlay to be rendered, for example:
′′′json
{
“frame_id”: 12345,
“rois”: [
{
“roi_id”: “roi-pancreas-tumor-01”,
“segmentation_mask”: [[x1,y1], [x2,y2], ...],
“classification_label”: “SOLID_MASS”,
“confidence_score”: 0.97,
“recommendation”: “BIOPSY_RECOMMENDED”
}

The cloud platform 108 is the computational core of the disclosed implementation. This component is responsible for executing the AI model and generating the diagnostic insights. The cloud platform 108 receives the multi-modal EUS video stream from the Client computing device, executes the trained AI model on each video frame or sequence of frames, performs identification, segmentation, and characterization of tissue, generates the result data (segmentation masks, classification labels) and transmits the result data back to the client device in real-time.

The cloud platform 108 can be implemented on a scalable cloud infrastructure (e.g., AWS, Google Cloud, Azure) by virtual machines or containers running on servers equipped with high-performance GPUs (e.g., NVIDIA A100, H100, or similar) optimized for deep learning inference. High-bandwidth internal networking and load balancers can be used to distribute incoming requests across the multiple GPUs.

The cloud platform 108 can be built as a set of interacting microservices (E.g., containerized using Docker and orchestrated with Kubernetes). This ensures scalability, resilience, and independent deployability of components. Key microservices can include, for example, an Ingestion Service (or receiving and preprocessing incoming data streams, an AI Inference Service (for executing the trained AI model on the preprocessed data provided by the Ingestion Service, and a Results Dispatch Service (for managing the transmission of AI-generated results back to the client computing device 104 or other system components).

The AI model 110 can be built and executed using standard deep learning frameworks such as PyTorch or TensorFlow. Python can be used as the programming language for AI model development and execution due to its extensive ecosystem of scientific computing and ML libraries. Services for API endpoints may use Go or Node. js for high concurrency. The AI model can be optimized for low-latency inference using tools like NVIDIA TensorRT to convert the trained model into a highly efficient runtime engine.

As described above, WebSockets can be used for real-time data exchange and REST/gRPC can be used for other API calls. Microservices communicate internally using lightweight protocols like gRPC for high performance or via a message queue (e.g., RabbitMQ or Kafka) for asynchronous task handling and decoupling.

The EMR module 412 can be configured to manage all persistent data, including AI models, training datasets, and integration with external clinical systems. This module is operative to store and manage the trained AI models and their versions, store the large, de-identified dataset of annotated EUS videos and corresponding “ground truth” data (pathology, genomics, etc.) for training and validation. The EMR data module 412 can also provide an interface to connect to external Electronic Medical Record (EMR) systems to retrieve non-imaging patient data and store newly generated data, such as AI analysis results or clinician feedback, for future retraining. For storing large binary files like videos and images, Amazon S3, Google Cloud Storage, or the like can be used. Various databases such as a relational database (e.g., PostgreSQL) or NoSQL databases (e.g., MongoDB or DynamoDB) can be used for structured and semi-structured metadata, patient information, and analysis results.

Integration with EMR systems (e.g., Epic or Cerner) can be achieved via standard healthcare interoperability protocols, such as Health Level Seven (HL7) and Fast Healthcare Interoperability Resources (FHIR). The EMR data module 412 can act as an API gateway, wrapping these complex protocols into a uniform internal REST or gRPC API for the AI Engine of Cloud Platform and AI Processing Engine to query.

For large-scale training, the data can be organized in a data lake architecture, allowing for flexible querying and processing by data science teams. Database tables can be used to link patients, procedures, video files, AI results, and ground-truth pathology data. When communicating with EMR systems, data can be structured according to FHIR resources, such as ‘Patient’, ‘Observation’ (for lab results), ‘DiagnosticReport’ (for pathology), and ‘ImagingStudy’.

The system can be configured to ensure compliance with healthcare regulations like HIPAA to protect sensitive patient data. Users (e.g., clinicians) can be authenticated and authorized for access to the system. Role-based access control (RBAC) can be enforced, ensuring users can only perform actions and view data appropriate to their role. All data can be encrypted in transit and at rest and all system access and actions can be securely logged for auditing purposes. Authentication can be implemented using standard protocols like OAuth 2.0 or OpenID Connect (OIDC) and can be integrated with a hospital's existing identity provider (e.g., Active Directory) via SAML. A dedicated authorization service can manage user roles and permissions. Permissions can be defined in a database and checked at the API gateway level for every incoming request. TLS 1.2/1.3 encryption can be used to encrypt all data in transit between the components and EMR systems. Data at rest in object storage and databases can be encrypted using industry-standard algorithms like AES-256, for example. All Protected Health Information (PHI) can be stripped from the data before it is used for AI model training, in compliance with HIPAA Safe Harbor or Expert Determination methods.

A database table or JSON configuration can be used to define roles and their associated permissions, for example:
′′′json
{
“role”: “clinician”,
“permissions”: [“view_patient_data”, “run_ai_analysis”],
“role”: “data_scientist”,
“permissions”: [“access_deidentified_training_data”, “train_model”]
}

′′′

Audit logs can be stored in a structured format (e.g., JSON) in a dedicated, immutable logging system (e.g., Amazon CloudWatch Logs, ELK Stack). Each entry includes a timestamp, user ID, action performed, and target resource.

The efficacy of the disclosed implementations is dependent on the robustness and accuracy of the underlying artificial intelligence (AI) model. Examples of processes for training, validating, and deploying the AI model to yield high performance in a clinical setting are set forth below.

The foundation of the training process acquisition and curation process of a comprehensive, multi-modal, and clinically-validated dataset is designed to create a rich repository of data that reflects the diversity of clinical scenarios. Data is collected retrospectively and prospectively from one or more clinical institutions. It is desirable to collect a complete data and document set for each patient case (which can be specified in a clinical protocol). An example data set includes:
Primary Imaging Data: Raw, high-fidelity Endoscopic Ultrasound (EUS) procedural videos and/or still images captured during clinical procedures. This data is multi-modal, and can include:
   B-Mode (Grey-Scale) Ultrasound: Provides anatomical and structural information.
   Elastography: Provides quantitative or qualitative data representing tissue stiffness, a critical feature for differentiating hard tumorous tissue from soft normal tissue.
   Color Doppler, Contrast-Enhanced Arterial Phase, and Contrast-Enhanced Venous Phase imaging.
Confirmatory Ground Truth Data: To ensure the AI model is trained against definitive clinical outcomes, the imaging data is linked to a “gold standard” of disease confirmation. This can include one or more of the following:
   Pathology Reports: Cytology and/or histology results from Fine-Needle Aspiration or Biopsy (FNA/FNB).
   Surgical Pathology: Confirmatory results from resected specimens (e.g., following a Whipple procedure).
Clinical Progression: Longitudinal imaging (e.g., CT/MRI) or clinical course that confirms malignancy (e.g., lesion growth, vascular invasion, metastatic spread).
Multidisciplinary Tumor Board Adjudication: A consensus diagnosis from a panel of experts.
Correlative Data: Additional data points are collected to enable the training of more sophisticated models and to provide a holistic patient view. This includes serum tumor markers (e.g., CA19-9, CEA), liquid biopsy results (e.g., ctDNA, KRAS mutations), and structured/unstructured data from the Electronic Medical Record (EMR).
   All collected data undergoes a rigorous de-identification process in compliance with HIPAA standards to remove any Protected Health Information (PHI) before being used for training.

All collected data can undergo a de-identification process in compliance with HIPAA standards to remove any Protected Health Information (PHI) before being used for training.

Once acquired, the raw imaging data can be annotated to create labeled examples for supervised machine learning. This process generates the “ground truth” that the AI model learns to replicate. The annotation process can be conducted by trained medical professionals, such as gastroenterologists, radiologists, or specialized medical annotators who review the EUS videos and still images using specialized annotation software. For each relevant frame or video segment, the annotators perform two primary tasks:
Segmentation: They draw precise polygonal outlines (segmentation masks) around regions of interest. These regions include anatomical structures (e.g., the pancreas itself) and pathological findings.
Labeling: Each segmented region is assigned a classification label based on the corresponding ground truth confirmatory data.

The primary classifications can include, but are not limited to:
CONFIRMED_TUMOR (for malignant neoplasms)
CONFIRMED_CYSTIC_LESION (for benign or indeterminate cysts like serous cystadenoma, IPMN, etc.)
NORMAL_TISSUE (for healthy pancreatic parenchyma)

This process results in a dataset where each image is paired with a corresponding mask and class label, forming the basis for training the segmentation and classification model. Before being fed into the AI model in a training phase, the data undergoes pre-processing and augmentation to standardize the input and thus enhance the robustness of the trained AI model:
Pre-processing: This includes steps such as:
   Normalization: Resizing all images to a uniform resolution (e.g., 512×512 pixels).
   Intensity Scaling: Normalizing pixel values across all modalities to a consistent range (e.g., [0, 1]).
   Artifact Removal: Applying filters to remove on-screen text, measurement overlays, or other visual artifacts from the EUS machine's output.
Augmentation: To artificially expand the size and diversity of the training dataset and prevent overfitting, various data augmentation techniques are applied. These include random geometric transformations (rotation, scaling, flipping, cropping) and photometric transformations (adjusting brightness, contrast, and saturation). This ensures the model learns to identify pathologies regardless of minor variations in image orientation, zoom, or lighting conditions.

While various architectures can be used for the AI model, an exemplary and highly effective architecture for this task is a U-Net or a similar encoder-decoder network. The U-Net architecture is particularly well-suited for biomedical image

segmentation. It consists of a contracting path (the “encoder”) that captures context and spatial hierarchies, and a symmetric expanding path (the “decoder”) that enables precise localization. “Skip connections” between the encoder and decoder paths allow the model to combine high-level feature information with fine-grained spatial details, which is essential for accurately delineating lesion boundaries. Alternative Architectures may include more advanced variants like U-Net++, or models designed for simultaneous detection and segmentation such as Mask R-CNN. For analyzing video data, the architecture may incorporate recurrent layers (e.g., LSTM) or temporal convolutions to leverage information across multiple frames.

The AI model can be designed to process the plurality of imaging modalities. This can be achieved by providing multiple input channels to the network, where each channel corresponds to a different modality (e.g., one channel for greyscale, one for elastography). The model learns to fuse the information from these channels to make a more informed prediction.

The model is trained by minimizing a loss function that quantifies the difference between the model's prediction and the ground truth annotation. For a combined segmentation and classification task, a composite loss function is typically used, such as a weighted sum of:
A segmentation loss, like Dice Loss or Jaccard (IoU) Loss, which is effective for handling class imbalance in image segmentation.
A classification loss, such as Categorical Cross-Entropy, for the pixel-wise classification task.

An optimization algorithm, such as Adam (Adaptive Moment Estimation) or AdamW, can be used to update the model's weights via backpropagation based on the calculated loss. The training is typically performed iteratively over many epochs. In each epoch, the model processes batches of training data, calculates the loss, and adjusts its parameters to minimize the loss. The process continues until the model's performance on a separate validation dataset converges.

The performance of the trained AI model can be evaluated on validation data (e.g., a held-out test dataset that was not used during training). Performance is measured using standard clinical and statistical metrics, including:
Sensitivity (Recall): The ability of the model to correctly identify true positive cases (e.g., correctly flagging a confirmed tumor).
Specificity: The ability of the model to correctly identify true negative cases (e.g., correctly identifying tissue as normal).
Dice Score/Jaccard Index: To measure the spatial overlap between the predicted segmentation mask and the ground truth mask.

The goal is to achieve high performance thresholds, such as ≥90% sensitivity and ≥85% specificity, to ensure clinical utility.

Once validated, the trained model can be optimized for efficient inference (e.g., using NVIDIA TensorRT) and deployed to the AI Processing Engine on the cloud platform. The system is designed for continuous improvement; as new clinical data is acquired and annotated, it can be added to the training dataset, and the model can be periodically retrained to enhance its accuracy and adapt to new data patterns. This creates a learning loop that ensures the model remains state-of-the-art.

Features illustrated or described as part of one implementation can be used in conjunction with features of other implementations. Further, it will be apparent to one of ordinary skill in the art that the systems and methods described herein may be applied to the analysis of various organs beyond the pancreas, and may incorporate additional imaging modalities or data sources not explicitly detailed. While specific software architectures, AI models, and communication protocols have been described, those skilled in the art will recognize that other functionally equivalent implementations are possible. While the invention has been described in connection with various implementations, it is to be understood that the disclosure is not to be limited to these implementations but include various modifications and equivalent arrangements included within the spirit and scope of the appended claims.

1. A computer-implemented method for real-time analysis of pancreatic tissue, the method comprising:
receiving, at a computing device, a real-time endoscopic ultrasound (EUS) video stream of a pancreas of a patient, wherein the EUS video stream comprises a plurality of imaging modalities including at least grey-scale data and elastography data representing tissue stiffness;
processing, by one or more processors using an artificial intelligence (AI) model, the EUS video stream to:
identify a region of interest within the pancreas;
segment the identified region of interest from surrounding tissue; and
generate a characterization of the segmented region of interest based on an analysis of the plurality of imaging modalities, including the elastography data; and
generating, for display on a user interface in real-time, a visual output comprising the EUS video stream with a graphical overlay, wherein the graphical overlay includes a visual indicator delineating the segmented region of interest and a classification label corresponding to the generated characterization.

2. The method of claim 1, wherein the plurality of imaging modalities further comprises one or more of color doppler data, contrast-enhanced arterial phase data, or contrast-enhanced venous phase data.

3. The method of claim 1, wherein the classification label indicates that the segmented region of interest is one of a solid mass, a cystic lesion, or normal tissue.

4. The method of claim 1, wherein processing the EUS video stream by the AI model is performed on one or more remote servers in a cloud-computing environment, and wherein the computing device receives the visual output from the one or more remote servers.

5. The method of claim 1, further comprising:
accessing a set of non-imaging patient data from an electronic medical record, the non-imaging patient data comprising at least one of laboratory test results, genomic data, or pathology reports; and
wherein the AI model refines the characterization of the segmented region of interest based on the accessed non-imaging patient data.

6. The method of claim 1, further comprising generating, based on the characterization of the segmented region of interest, a recommendation for further procedures to be conducted on the segmented region of interest based on the characterization of the segmented region of interest.

7. A system for real-time analysis of pancreatic tissue, the system comprising:
one or more processors; and
a memory storing instructions that, when executed by the one or more processors, cause the system to perform a method comprising:
receiving, at a computing device, a real-time endoscopic ultrasound (EUS) video stream of a pancreas of a patient, wherein the EUS video stream comprises a plurality of imaging modalities including at least grey-scale data and elastography data representing tissue stiffness;
processing, by one or more processors using an artificial intelligence (AI) model, the EUS video stream to:
identify a region of interest within the pancreas;
segment the identified region of interest from surrounding tissue; and
generate a characterization of the segmented region of interest based on an analysis of the plurality of imaging modalities, including the elastography data; and
generating, for display on a user interface in real-time, a visual output comprising the EUS video stream with a graphical overlay, wherein the graphical overlay includes a visual indicator delineating the segmented region of interest and a classification label corresponding to the generated characterization.

8. The system of claim 7, wherein the plurality of imaging modalities further comprises one or more of color doppler data, contrast-enhanced arterial phase data, or contrast-enhanced venous phase data.

9. The system of claim 7, wherein the classification label indicates that the segmented region of interest is one of a solid mass, a cystic lesion, or normal tissue.

10. The system or claim 7, wherein processing the EUS video stream by the AI model is performed on one or more remote servers in a cloud-computing environment, and wherein the computing device receives the visual output from the one or more remote servers.

11. The system of claim 7, wherein the method further comprises:
accessing a set of non-imaging patient data from an electronic medical record, the non-imaging patient data comprising at least one of laboratory test results, genomic data, or pathology reports; and
wherein the AI model refines the characterization of the segmented region of interest based on the accessed non-imaging patient data.

12. The system of claim 7, wherein the method further comprises generating, based on the characterization of the segmented region of interest, a recommendation for further procedures to be conducted on the segmented region of interest based on the characterization of the segmented region of interest.

13. Non-transitory computer-readable media have instructions stored thereon that, when executed by one or more computer processors, cause the one or more processors to perform a method comprising:
receiving, at a computing device, a real-time endoscopic ultrasound (EUS) video stream of a pancreas of a patient, wherein the EUS video stream comprises a plurality of imaging modalities including at least grey-scale data and elastography data representing tissue stiffness;
processing, by one or more processors using an artificial intelligence (AI) model, the EUS video stream to:
identify a region of interest within the pancreas;
segment the identified region of interest from surrounding tissue; and
generate a characterization of the segmented region of interest based on an analysis of the plurality of imaging modalities, including the elastography data; and
generating, for display on a user interface in real-time, a visual output comprising the EUS video stream with a graphical overlay, wherein the graphical overlay includes a visual indicator delineating the segmented region of interest and a classification label corresponding to the generated characterization;
Based on the provided excerpt from the patent application, here are several suggestions to improve clarity, completeness, and enforceability.

14. The media of claim 13 wherein the plurality of imaging modalities further comprises one or more of color doppler data, contrast-enhanced arterial phase data, or contrast-enhanced venous phase data.

15. The media of claim 13, wherein the classification label indicates that the segmented region of interest is one of a solid mass, a cystic lesion, or normal tissue.

16. The media of claim 13, wherein processing the EUS video stream by the AI model is performed on one or more remote servers in a cloud-computing environment, and wherein the computing device receives the visual output from the one or more remote servers.

17. The media of claim 13, wherein the method further comprises:
accessing a set of non-imaging patient data from an electronic medical record, the non-imaging patient data comprising at least one of laboratory test results, genomic data, or pathology reports; and
wherein the AI model refines the characterization of the segmented region of interest based on the accessed non-imaging patient data.

18. The media of claim 13, wherein the method further comprises generating, based on the characterization of the segmented region of interest, a recommendation for further procedures to be conducted on the segmented region of interest based on the characterization of the segmented region of interest.
